SEQUENCE LISTING (11 sequences)

SEQ ID NO: 1 - protein, 14 aa, Artificial Sequence
  Synthetic consensus peptide
XXXWVXXXXXXXXX

SEQ ID NO: 2 - protein, 14 aa, Homo sapiens
DSFWVKNELLPNLE

SEQ ID NO: 3 - protein, 14 aa, Homo sapiens
DAYWVENLMVQELE

SEQ ID NO: 4 - protein, 14 aa, Homo sapiens
DKDWVWEHFSSMEK

SEQ ID NO: 5 - protein, 14 aa, Homo sapiens
DEDWVRNELVKNLE

SEQ ID NO: 6 - protein, 14 aa, Homo sapiens
DFTWVQNALLKHLD

SEQ ID NO: 7 - protein, 14 aa, Homo sapiens
DSAWVKSELVPYLE

SEQ ID NO: 8 - protein, 14 aa, Homo sapiens
VTEWVLAELVAKLE

SEQ ID NO: 9 - protein, 14 aa, Homo sapiens
VTDWVINELRYHLE

SEQ ID NO: 10 - protein, 14 aa, Homo sapiens
VADWVYNELRGQLE

SEQ ID NO: 11 - protein, 8 aa, Artificial Sequence
  Synthetic peptide
SIINFEKL

(19) United States
(12) Patent Application Publication
(10) Pub. No.: US 2008/0233105 A1
(43) Pub. Date: Sep. 25, 2008

(54) Compositions and methods for preventing or treating a viral infection

(76) Inventors: William R. Green, Etna, NH (US); Kathy A. Green, Etna, NH (US); Edward J. Usherwood, Hanover, NH (US)

Correspondence Address: Jane Massey Licata;Licata & Tyrrell P.C., 66 E. Main Street, Marlton, NJ, 08053, US

(21) Appl. No.: 11/519,408

(22) Filed: Sep. 12, 2006

Related U.S. Application Data
(60) Provisional application No. 60/716,752, filed on Sep. 13, 2005.

(51) Int. Cl. A61K 39/395; A61K 39/00; A61P 37/00

(52) U.S. Cl. 424/130.1; 424/204.1

(57) ABSTRACT

The present invention is a composition for enhancing the immunogenicity of viral vaccine. The composition encompasses a viral vaccine in combination with at least one toll-like receptor and, in particular embodiments, an anti-CD40 antibody. The compositions of the instant invention find application in the prevention or treatment of a viral infection.

This invention was made in the course of research sponsored by the National Institute of Allergy and Infectious Diseases (Grant No. AI 1057159). The U.S. government may have certain rights in this invention.

### INTRODUCTION

This application claims benefit of U.S. Provisional Patent Application Ser. No. 60/716,752 filed Sep. 13, 2005, the content of which is incorporated herein by reference in its entirety.

### BACKGROUND OF THE INVENTION

Front-line, anti-microbial defense is accomplished by the innate immune system with the help of pattern recognition receptors, such as the Toll-like receptors (TLRs), in early detection of specific classes of pathogens (Janeway and Medzhitov (2002) Ann. Rev. Immunol. 20:197; Barton and Medzhitov (2002) Curr. Top. Microbiol. Immunol. 270:81; Medzhitov (2001) Nat. Rev. Immunol. 1:135; Heine and Lein (2003) Int. Arch. Allergy Immunol. 130:180). The broad classes of pathogens (e.g., viruses, bacteria, and fungi) constitutively express a set of class-specific, mutation-resistant molecules called pathogen-associated molecular patterns (PAMPs). These microbial molecular markers are composed of proteins, carbohydrates, lipids, nucleic acids and/or combinations thereof, and are located internally or externally.

Pattern recognition receptors are constitutively expressed to allow the host to detect the pathogen regardless of its life cycle stage. Further, such receptors are mutation resistant, allowing the host to recognize the pathogen regardless of its particular strain (Janeway and Medzhitov (2002) supra; Barton and Medzhitov (2002) supra; Medzhitov (2001) supra; Gordon (2002) Cell 111:927). Pattern recognition receptors do more than merely recognize pathogens via their PAMPs. Once bound, pattern recognition receptors tend to cluster, recruit other extracellular and intracellular proteins to the complex, and initiate signaling cascades that ultimately impact transcription (Janeway and Medzhitov (2002) supra; Medzhitov (2001) supra; Heine and Lein (2003) supra). Further, pattern recognition receptors are involved in activation of complement, coagulation, phagocytosis, inflammation, and apoptosis functions in response to pathogen detection (Janeway and Medzhitov (2002) supra; Barton and Medzhitov (2002) supra; Medzhitov (2001) supra). There are several types of pattern recognition receptors including complement, glucan, mannose, scavenger, and TLR, each with specific PAMP ligands, expression patterns, signaling pathways, and anti-pathogen responses (Janeway and Medzhitov (2002) supra; Gordon (2002) supra; Modlin (2002) Ann. Allergy Asthma Immunol. 88:543).

The TLR family has been described as type I transmembrane pattern recognition receptors that possess varying numbers of extracellular N-terminal leucine-rich repeat motifs, followed by a cysteine-rich region, a transmembrane domain, and an intracellular Toll/IL-1 R (TIR) motif (Hashimoto, et al. (1988) Cell 52:269; Medzhitov, et al. (1997) Nature 388:394; Rock, et al. (1998) Proc. Natl. Acad. Sci. USA 95:588; Chaudhary, et al. (1998) Blood 91:4020; Takeuchi, et al. (1999) Gene 231:59; Chuang and Ulevitch (2001) Eur. Cytokine Netw. 11:372; Du, et al. (2000) Eur. Cytokine Netw. 11:362). The leucine-rich repeat domain is important for ligand binding and associated signaling and is a common feature of pattern recognition receptors (Modlin (2002) supra; Kobe and Deisenhofer (1995) Curr. Opin. Struct. Biol. 5:409). The TIR domain is important in protein-protein interactions and is typically associated with innate immunity (Aravind, et al. (2001) Science 291:1279).

U.S. patent application Ser. No. 11/026,457 discloses TLR6, TLR7, TLR8, and TLR9 agonists as adjuvants for inducing a systemic immune response, a localized immune response, or both to treat viral infections. This reference further teaches that immune responses can be augmented by the co-administration of cytokines such as CD40 ligand.

U.S. patent application Ser. No. 11/184,065 teaches immune stimulating complexes containing an inert TLR ligand in combination with sterol or saponin for use in inducing innate immunity and the treatment of viral infections. This reference further teaches the co-administration of cytokines such as CD40 ligand.

### SUMMARY OF THE INVENTION

The present invention is a composition composed of a viral vaccine and at least one Toll-like receptor agonist. In one embodiment, the composition further contains an anti-CD40 antibody. In other embodiments the Toll-like receptor being agonized is an intracellular receptor. In still further embodiments, the composition contains at least two Toll-like receptor agonists.

The present invention is also a method for increasing the immunogenicity of viral vaccine. The method involves administering a viral vaccine in combination with at least one Toll-like receptor agonist and, in particular embodiments an anti-CD40 antibody, thereby increasing the immunogenicity of the viral vaccine.

A method for preventing or treating a viral infection is also provided. Prevention or treatment is accomplished by administering an effective amount of a viral vaccine in combination with at least one Toll-like receptor agonist and, in particular embodiments an anti-CD40 antibody.

### BRIEF DESCRIPTION OF THE DRAWINGS

FIG. 1 depicts the TLR signaling pathway, wherein TIR domain-containing adaptors, such as MyD88, TIRAP/Mal, TRIF, and TRAM, regulate TLR-mediated signaling pathways. MyD88, which is common to all TLR-mediated pathways with the exception of TLR3, leads to the production of inflammatory cytokines, whereas TRIF mediate induction of IFN-gamma in TLR3 and TLR4 signaling pathways. TIRAP/Mal is implicated in the TLR2- and TLR-4 mediated MyD88-dependent signaling pathway. TRAM is specifically involved in the TLR4-mediated TRIF-dependent pathway.

### DETAILED DESCRIPTION OF THE INVENTION

Novel compositions and methods for increasing both primary and memory cytolytic T lymphocytes (CTL) responses as well as IFN-gamma production and neutralizing antibody responses to a weakly immunogenic viral vaccine have now been found. Such compositions and methods involve combining a noninfectious or attenuated viral vaccine with at least one toll-like receptor (TLR) agonist as an adjuvant to enhance the immunogenicity of the viral vaccine. By stimulating immune responses to viral vaccines, the compositions and methods of the present invention find application in the prevention and treatment of viral infections. The instant compositions and methods are particularly effective for the acute primary CTL response needed for “ring immunization” to geographically-defined outbreaks. Moreover, given the efficacy of the instant compositions practical problems associated with the decreased participation with respect to revisits to the clinic for needed booster immunizations can be circumvented.

To illustrate the effectiveness and efficacy of the instant invention, TLR agonists were co-administered with an attenuated, modified, replication-deficient Ankara strain (MVA) of vaccinia virus and immune responses in mice were analyzed. It was found that attenuated MVA when used in combination with one or more TLR agonists, with or without an anti-CD40 antibody, could elicit a primary and memory CTL immune response which was comparable to the Western Reserve strain of vaccinia virus, a replication competent strain. These results demonstrate that a single concurrent injection or co-injection of TLR agonist with conventional viral vaccine can increase the immunogenicity of said vaccine. Thus, the instant invention is a composition containing a noninfectious or attenuated viral vaccine in combination with at least one TLR agonist and, in particular embodiments an anti-CD40 antibody, for use in methods for increasing the immunogenicity of the viral vaccine, and preventing or treating viral infection.

In the context of the present invention, a viral vaccine encompasses noninfectious or attenuated viral vaccines, which are less immunogenic than their live, infectious, or replication-competent counterparts. As used herein, an attenuated viral vaccine, refers to a virus which is capable of infecting a host cell, but has either significantly diminished or no capacity to cause disease in an animal. An attenuated viral vaccine can be generated by, e.g., mutation or cold-adaptation (Maassab & DeBorde (1985) Vaccine 3:355-369).

Noninfectious viral vaccines include inactivated killed vaccines, subunit vaccines, synthetic peptide and biosynthetic polypeptide vaccines, oral transgenic plant vaccines, anti-idiotype antibody vaccines, DNA vaccines, and polysaccharide-protein conjugate vaccines which are incapable of infecting and replicating in a host cell and are also largely incapable of causing disease in an animal.

The term “vaccine” as used herein is meant an antigen or a bioactive agent, e.g., a virus or immunogenic protein, that elicits an immune response in a subject to which the vaccine has been administered. In one embodiment, the immune response confers some beneficial, protective effect to the subject as against a subsequent challenge with the same or a similar bioactive agent. More desirably, the immune response prevents the onset of or ameliorates at least one symptom of a disease associated with the bioactive agent, or reduces the severity of at least one symptom of a disease associated with the bioactive agent upon subsequent challenge. Even more desirably, the immune response prevents the onset of or ameliorates more than one symptom of a disease associated with the bioactive agent upon subsequent challenge.

In another embodiment, the immune response confers a beneficial, therapeutic effect to a subject already infected with a viral pathogen (e.g., HIV-infected subjects or overt AIDS patients). In this regard, the immune response ameliorates one or more symptoms of a viral disease or reduces viral load.

A viral vaccine of the present invention is desirably an attenuated viral vaccine or noninfectious viral vaccine; however, combinations of these vaccines, or any bioactive agent eliciting a CD8+ cell and/or antibody response, are also contemplated by the present invention. In particular embodiments, the present invention embraces viral vaccines to variola virus, vaccinia virus, HIV, or influenza virus.

Variola virus, the most virulent member of the genus Orthopoxvirus, specifically infects humans and causes smallpox. Smallpox has been designated as a category A biological weapon because it is easily transmittable, has a high mortality rate, would likely cause panic and social disruption, and requires special action for public health preparedness. Following an incubation period, infected persons have prodromal symptoms that include high fever, back pain, malaise, and prostration. The eruptive stage is characterized by maculopapular rash that progresses to papules, then vesicles, and then pustules and scab lesions. The mortality rate for smallpox is approximately 30%. Patients having a fever and rash may be confused with having chickenpox. The most effective method for preventing smallpox epidemic progression is vaccination. The conventional vaccine is a live vaccinia virus preparation administered by scarification with a bifurcated needle. The immune response is protective against orthopoxviruses, including variola. Vaccination is associated with moderate to severe complications, such as generalized vaccinia, eczema vaccinatum, progressive vaccinia, and post-vaccinial encephalitis. Efforts for vaccine production have focused on a live cell culture-derived vaccinia virus vaccine, subunit designs and the use of other vectors. In particular embodiments, a modified vaccinia Ankara (MVA) strain is embraced by the instant invention. Desirably a strain of vaccinia Ankara which is replication incompetent and has attenuated virulence is employed. Suitable MVA strains for use in accordance with the instant invention are well-known to those of skill in the art.

Two types of influenza vaccines are conventionally employed. The first type is an inactivated vaccine composed of purified virus grown in embryonated hen's eggs. Following purification, the virus is inactivated with formaldehyde and treated with detergent to release the immunogenic surface antigens (hemaggutinin and neuraminidase). Detergent ‘splitting’ of the virus also reduces the fever associated with vaccine administration (pyrogenicity). The second type is an attenuated vaccine, adapted to grow at colder temperatures than the human respiratory tract, which is not pathogenic in humans (Maassab & DeBorde (1985) Vaccine 3(5):355-369). While the inactivated vaccine is administered as an intramuscular injection, the attenuated vaccine is administered in the nose, allowing local respiratory immunity to be generated. Other vaccines of use include genetically engineered attenuated vaccines or purified components of viral proteins (Sheridan (2004) Nat. Biotechnol. 22(12):1487-8).

While these vaccines induce adequate immunity to infection, protection appears to be only short lived, so a new vaccine is required each year. Another shortcoming of the current vaccines is that they generally provide immunity only to the specific viral serotypes included in the vaccine. As the serotypes in circulation constantly change, there is a need to re-vaccinate each year with the appropriate serotypes.

Using a composition of the present invention, limitations of conventional influenza viral vaccines are overcome. For example, as the antibody and T cell responses are enhanced relative to the vaccine alone, the memory response will also be enhanced, leading to longer-term immunity. Further, T cell responses to conserved viral proteins are be enhanced with this approach, leading to greater cross-serotype protection. Moreover, because the instant composition magnifies the immune response, vaccine dose can be reduced, allowing scarce supplies of vaccine to protect a larger number of individuals.

In accordance with the present invention, the type of virus to be used in a vaccine is desirably influenza virus type A, although other influenza viruses that are known, or are as yet unknown, are also included in the invention. There presently exists a number of different serotypes of influenza virus type A, and their ability to cause disease and induce immunity in humans and other animals is governed in large part by the type of HA and NA antigens in the envelope of the virus. The present invention should be construed to include any and all viruses having any and all combinations of HA and NA antigens in the viral envelope, irrespective of whether these virus strains are produced during natural infection of a host, are produced by reassortment of HA and NA antigens as a result of infection of different species, or are produced by recombinant means where the antigenic make up of the virus is either specifically designed or is generated by random recombination as is possible using ordinary molecular biology techniques. An influenza viral vaccine useful in the invention is one that is capable of eliciting a broad spectrum CD8+ T cell and/or antibody response in a subject. Desirably, the influenza viral vaccine is protective against an influenza virus including, but not limited to, those of potential pandemic strains of influenza virus (for example, H3N2, H5N1, H9N2, H7N, H7N2, H7N3 or H7N7), past pandemics (for example H2N2 or H1N1), or non-pandemic viruses (for example H1N1, H1N2 or H3N2). See Webby & Webster (2003) Science 302:1519-1522 and Sheridan (2004) Nat. Biotechn. 22:1487-88 for examples of influenza viral vaccines.

HIV/AIDS prevention and treatment has been hindered by the following: the propensity of the virus to mutate and create variant HIV with functionally disrupted epitopes, in particular, both in the viral epitopes per se and adjacent areas corresponding to antibody neutralization sites, and T-cell epitopes; and especially for therapeutic vaccines, the destruction of CD4 T cells. Vaccines to elicit cell-mediated immunity, particularly CD8+ T cell lytic (CTL) and cytokine-producing responses have been suggested. Rather than an endpoint of sterilizing immunity, these vaccines aim to decrease the viral load dramatically, converting HIV/AIDS into a much less severe, chronic illness, thereby substantially reducing the efficiency of person-to-person transmission of the virus (Girard & Osmanov (2006) supra 24:4062-4081; McMichael (2006) supra; Duerr, et al. (2006) Clin. Infect. Dis. 43:500-511). Vaccines of this type include an array of antigen preparations, vectors/vehicles, in various combinations, and particularly using two (or more) sequential immunizations with different preparations, i.e., the “prime/boost” regimen. Given that the instant composition elicits a neutralizing antibody response, CD8 T cell lytic activity and IFN-gamma production, the instant composition finds application in the protection or control of HIV infections. For example, HIV vaccines such as a recombinant MVA encoding HIV-1 antigenic determinants can be administered in combination with a TLR agonist(s) and anti-CD40 monoclonal antibody to provide substantially augmented responses.

Boosting the immune response to HIV-1 with the instant invention overcomes many of the most important limitations of the current vaccines in several ways. First, stronger initial responses, whether elicited by a single injection of antigen or a prime/boost strategy, generally lead to more vigorous and longer term memory responses. Second, a more robust response frequently allows for the generation of immune responses, both T-cell and neutralizing antibody, to the more weakly immunogenic but conserved viral epitopes, rather than just to the more highly immunogenic, strain-specific determinants that are so variable between HIV-1 viral isolates and within an isolate over a period of time. Development of strong immunity to these conserved epitopes leads to greater cross-serotype protection, which is very important to counter both the many different pre-existing antigenic forms of HIV-1 and its propensity to recombine and mutate under immune selective pressure. Third, the use of anti-CD40 monoclonal antibody allows for the functional replacement of the loss of CD4 T cells in AIDS. Thus, much of the loss of CD4 T-cell function can be ascribed to the concurrent loss of CD154 (CD40 ligand) which binds to CD40 on B cells to stimulate antibody production and on professional antigen presenting cells to greatly augment (together with stimulation through their TLR receptors) functional antigen presentation to antiviral T cells. Thus, the instant methods would not only be of benefit for prophylactic vaccine development but also vaccines devised to be used for AIDS patients after interruption of HAART therapy or in other settings whereby AIDS patients are vaccinated.

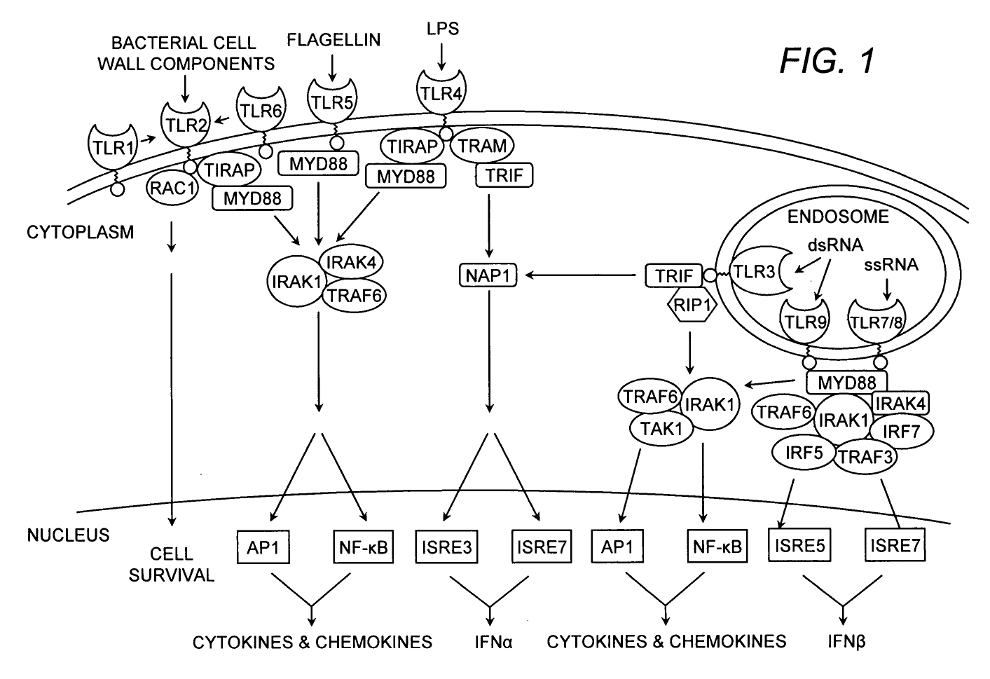

Eleven TLRs, named TLR1 to TLR11, have been identified in humans, and equivalent forms of many of these have been found in other mammalian species. Human TLR proteins are known in the art and provided under GENBANK Accession Nos. U88540 (TLR1; Rock, et al. (1998) supra), U88878 (TLR2; Rock, et al. (1998) supra), U88879 (TLR3; Rock, et al. (1998) supra), U88880 (TLR4; Medzhitov, et al. (1997) supra), AF051151 (TLR5; Chaudhary, et al (1998) supra), AB020807 (TL6), AF240467 (TLR7), AF245703 (TLR8), AF259262 (TLR9), and AF296673 (TLR10). All TLRs have a cytoplasmic signaling domain called the Toll/interleukin 1 receptor resistance (TIR) domain (Table 1), which associates with intracellular TIR domain-containing adaptors, such as MyD88, TIRAP, TRIF/TICAM1, and TRAM/TICAM2. These TLR-associated adaptor molecules in turn mediate downstream signaling to induce pro-inflammatory and/or anti-viral innate immune responses (Akira & Takeda (2004) Nat. Rev. Immunol. 4:499-511). See FIG. 1.

TABLE 1

| | | |
|---|---|---|
| | | SEQ |
| | | ID |
| TLR | TIR Motif Core Sequence | NO: |

| | | |
|---|---|---|
| TLR1 | Asp-Ser-Phe-Trp-Val-Lys-Asn-Glu-Leu-Leu- | 2 |
| | Pro-Asn-Leu-Glu | |
| TLR2 | Asp-Ala-Tyr-Trp-Val-Glu-Asn-Leu-Met-Val- | 3 |
| | Gln-Glu-Leu-Glu | |
| TLR3 | Asp-Lys-Asp-Trp-Val-Trp-Glu-His-Phe-Ser- | 4 |
| | Ser-Met-Glu-Lys | |
| TLR4 | Asp-Glu-Asp-Trp-Val-Arg-Asn-Glu-Leu-Val- | 5 |
| | Lys-Asn-Leu-Glu | |
| TLR5 | Asp-Phe-Thr-Trp-Val-Gln-Asn-Ala-Leu-Leu- | 6 |
| | Lys-His-Leu-Asp | |
| TLR6 | Asp-Ser-Ala-Trp-Val-Lys-Ser-Glu-Leu-Val- | 7 |
| | Pro-Tyr-Leu-Glu | |
| TLR7 | Val-Thr-Glu-Trp-Val-Leu-Ala-Glu-Leu-Val- | 8 |
| | Ala-Lys-Leu-Glu | |
| TLR8 | Val-Thr-Asp-Trp-Val-Ile-Asn-Glu-Leu-Arg- | 9 |
| | Tyr-His-Leu-Glu | |
| TLR9 | Val-Ala-Asp-Trp-Val-Tyr-Asn-Glu-Leu-Arg- | 10 |
| | Gly-Gln-Leu-Glu | |
| Cons. | Xaa₁-(Xaa₂)₂-Trp-Val-(Xaa₃)₃-Xaa₄-(Xaa₅)₃- | 1 |
| | Xaa₆-Xaa₇ | |

Xaa₁ denotes Val or Asp; Xaa₂, Xaa₃, and Xaa₅, denote any amino acid residue; Xaa₄ denotes Leu, Met or Phe; Xaa₆ denotes Leu or Glu; and Xaa₇ denotes Glu, Lys, or Asp.

While all TLRs are typical type I transmembrane proteins composed of an NH₂-terminal signal peptide, an extracellular domain involved in ligand recognition, a single transmembrane domain, and a cytoplasmic domain, it has been found that TLR1, TLR2, TLR4, TLR5, and TLR6 are expressed on the cell surface, whereas TLR3, TLR7, and TLR9 are localized in intracellular acidic compartments (Nishiya & DeFranco (2004) J. Biol. Chem. 279:19008-19017; Funami, et al. (2004) Int. Immunol. 16:1143-1154; Matsumoto, et al. (2003) J. Immunol. 171:3154-3162; Lee, et al. (2003) Proc. Natl. Acad. Sci. USA 100:6646-6651; Latz, et al. (2004) Nat. Immunol. 5:190-198; Zhang, et al. (2002) FEBS Lett. 532:171-176). Based on data with chimeric receptors, TLR8 appears to be localized primarily intracellularly but with a small fraction on the cell surface (Nishiya & DeFranco (2004) supra).

Because the specificity of TLRs cannot be changed, these receptors must recognize patterns that are constantly present on threats, not subject to mutation, and highly specific to threats (i.e., not normally found in the host where the TLR is present). Patterns that meet this requirement are usually critical to the pathogen's function and cannot be eliminated or changed through mutation; they are said to be evolutionarily conserved. Well-conserved features in pathogens include bacterial cell-surface lipopolysaccharides (LPS), lipoproteins, lipopeptides and lipoarabinomannan; proteins such as flagellin from bacterial flagella; double-stranded RNA of viruses or the unmethylated CpG islands of bacterial and viral DNA; and certain other RNA and DNA (see Table 2).

TABLE 2

| | | | Activation |
|---|---|---|---|
| Receptor | Ligand PAMP(s) | Localization | Cascade(s) |
| TLR1 | triacyl | cell surface | unknown |
| | lipoproteins | | |
| TLR2 | lipoproteins; gram | cell surface | MyD88-dependent |
| | positive | | TIRAP |
| | peptidoglycan; | | |
| | lipoteichoic acids; | | |
| | fungi; viral | | |
| | glycoproteins | | |
| TLR3 | double-stranded RNA | intracellular | MyD88- |
| | (as found in | | independent |
| | certain viruses), | | TRIF/TICAM |
| | poly I:C | | |
| TLR4 | lipopolysaccharide; | cell surface | MyD88-dependent |
| | viral glycoproteins | | TIRAP; MyD88- |
| | | | independent |
| | | | TRIF/TICAM/TRAM |
| TLR5 | flagellin | cell surface | MyD88-dependent |
| | | | IRAK |
| TLR6 | diacyl lipoproteins | cell surface | unknown |
| TLR7 | small synthetic | intracellular | MyD88-dependent |
| | compounds; single- | | IRAK |
| | stranded RNA | | |
| TLR8 | small synthetic | Intracellular/ | MyD88-dependent |
| | compounds; single- | cell surface | IRAK |
| | stranded RNA | | |
| TLR9 | unmethylated CpG | intracellular | MyD88-dependent |
| | DNA | | IRAK |

The Toll/interleukin-1 receptor (TIR) homology domain is an intracellular signaling domain found in MyD88, interleukin 1 receptor and the Toll-like receptors. It contains three highly-conserved regions, and mediates protein-protein interactions between the Toll-like receptors (TLRs) and signal-transduction components. When activated, TIR domains recruit cytoplasmic adaptor proteins MyD88 (GENBANK Accession No. Q99836) and TOLLIP (Toll interacting protein, GENBANK Accession No. Q9HOE2). In turn, these associate with various kinases to set off signaling cascades (Armant & Fenton (2002) Genome Biol. 3:3011}.

It has now been unexpectedly found that when a viral vaccine is administered with a TLR agonist, independent of whether the targeted TLR recognizes bacterial cell wall/surface components or pathogen nucleic acids, IFN-gamma production, CTL responses and neutralizing antibody responses to the viral vaccine are increased or enhanced. Thus, the instant invention embraces increasing the immunogenicity of a viral vaccine by combining the vaccine with any TLR agonist including those disclosed herein (e.g., PGN, CPG, pIC, LPS, imiquimod), as well as any other well-known agent (e.g., Malp-2, lipoarabinomannan, zymosan, modulin, taxol, resiquimod) which agonizes a toll-like receptor including, but not limited to, TLR1, TLR2, TLR3, TLR4, TLR5, TLR6, TLR7, TLR8, TLR9 or TLR10. Exemplary TLR agonists and their respective Toll-like receptors are listed in Table 3.

TABLE 3

| TLR | Agonist^{a} |
|---|---|
| TLR2 with TLR1 | Pam3CSK4 |
| TLR2 | HKLM, Lipomannan M. smegmatis, LPS |
| | P. gingivalis, LTA S. aureus, PGN S. aureus |
| TLR3 | Poly(I:C) |
| TLR4 | LPS E. coli K12 |
| TLR5 | Flagellin S. typhimurium |
| TLR6 with TLR2 | FSL1 |
| TLR7 | Imiquimod, Gardiquimod, Loxoribine |
| TLR8 | ssRNA40, PolyU/LyoVec |
| TLR9 | ODN2006, E. coli ssDNA/LyoVec, ODN2216 |

^{a}Agonists commercially available from INVIVOGEN (San Diego, CA).

While some embodiments embrace at least one TLR agonist, other embodiments embrace the use of at least two, three, four or more TLR agonists. In still other embodiments, when at least two or more TLR agonists are employed, the agonists are to different TLRs (e.g., TLR3 and TLR9). In yet other embodiments, the TLR agonist is to an intracellular TLR (i.e., TLR3, TLR7, TLR8, or TLR9). In still other embodiments, at least one TLR agonist to a MyD88-independent TLR is employed (e.g., TLR3).

Advantageously, it has also been appreciated that an anti-CD40 antibody can augment the immune response elicited by the viral vaccine and TLR agonist. Therefore, particular embodiments embrace the use of an anti-CD40 antibody in the compositions and methods of the present invention. Use of an anti-CD40 antibody for CD40 stimulation offers the advantages of protease resistance of the antibody and high intrinsic binding affinity and avidity for CD40. While an anti-CD40 monoclonal antibody is exemplified herein, the instant invention embraces the use of agonistic monoclonal or polyclonal antibodies to CD40, as well as agonistic fragments thereof. Desirably, the anti-CD40 antibody of the invention delivers a stimulatory signal through CD40 and/or increases the interaction between CD40 and CD40 ligand. Exemplary anti-CD40 antibodies include, but are not limited to, G28-5 (U.S. Pat. No. 5,182,368); CD40.4 (5C3) (PHARMINGEN, San Diego, Calif.); S2C6 (Paulie, et al. (1989) J. Immunol. 142:590-595); and recombinant S2C6 (U.S. Pat. No. 6,946,129). As used in the context of the present invention, an agonistic fragment of an anti-CD40 antibody retains the ability to recognize CD40 and includes F(ab′)₂ fragments, which can be produced by pepsin digestion of the antibody molecule, and the F(ab′) fragments, which can be generated by reducing the disulfide bridges of the F(ab′)₂ fragments. Alternatively, Fab expression libraries can be constructed (Huse, et al. (1989) Science 246:1275-1281) to allow rapid and easy identification of monoclonal Fab fragments with the desired specificity. In particular embodiments, a TLR3 and/or TLR9 agonist is used in combination with an anti-CD40 monoclonal antibody.

Because vaccines employing attenuated or noninfectious viral vaccines in combination with a TLR agonist are significantly less toxic and replication incompetent, such vaccines pose a reduced threat to the general population and in particular immunosuppressed subjects. Accordingly, the compositions disclosed herein can be used in the prevention or treatment of a viral infection, e.g., HIV, influenza, or variola or related poxvirus infection, in healthy and immunosuppressed individuals or to diminish viral pathogenesis.

TLR agonists of the present invention are prophylactically or therapeutically useful as they enhance or increase the immunogenicity of a viral vaccine. As used in the context of the instant invention, increasing the immunogenicity of a viral vaccine is intended to mean that antibody responses, especially neutralizing antibody production; IFN-gamma production; CD4 T-cell responses, both helper responses for maximal development of B-cell and CD8 T-cell immnunity, and CD4 T-effector cell responses per se; and/or direct stimulation of CD8 CTL responses are increased. In particular embodiments, immune responses to inherently weaker, but more conserved, cross-reactive epitopes are substantially enhanced when compared to administration of the viral vaccine alone. The ability to generate such cross-reactive responses is relevant to both seasonal flu in providing a strategy to counter the rapidly evolving variations of antigens, and to avian strains because of the low immunogenicity of conventional vaccines. In particular embodiments, immunogenicity is increased by at least 4-fold, 5-fold, 10-fold, or 40-fold.

In the context of prevention (i.e., primary prophylaxis) or treatment (i.e., secondary prophylaxis), an effective amount of a viral vaccine is administered with at least one TLR agonist and, in particular embodiments an anti-CD40 antibody, so that a viral infection is prevented or treated. Primary prophylaxis is achieved by administering a composition of the present invention to a subject in order to prevent infection, whereas secondary prophylaxis is employed when a subject has already been exposed to a pathogenic virus and has not yet become ill or is receiving some form of conventional antiviral therapy to alleviate signs or symptoms of a viral infection. For example, it is known that CD8+ T cells are responsible for control of HIV viral load (McMichael (2006) Annu. Rev. Immunol. 24:227-255). Thus, it is contemplated that the instant composition can be employed during a structured treatment interruption in HIV-1-infected subjects receiving highly active antiretroviral therapy (HAART). It has been found that structured treatment interruptions of 1-month duration separated by 1 month of HAART, before a final 3-month structured treatment interruption, results in augmented CD8⁺ T cell responses (Ortiz, et al. (2001) Proc. Natl. Acad. Sci. USA 98:13288-13293). Administration of a composition of the present invention during a structured treatment interruption can be used to elicit a CTL and neutralizing antibody response to common HIV epitopes, so that the HIV viral load is reduced. Accordingly, in certain embodiments, the instant composition is administered as a secondary prophylaxis. In particular embodiments, the administration of a composition of the present invention is carried out during a structured treatment interruption of antiviral therapy.

As used in the context of the present invention, administration of a viral vaccine with a TLR agonist and an anti-CD40 antibody, means that the TLR agonist and anti-CD40 antibody can be administered prior to, concurrently with, or after administration or vaccination with a viral vaccine. Desirably, administration of the TLR agonist and anti-CD40 antibody is within 5 minutes, 30 minutes, 1 hour, or 2 hours of vaccine administration. Further, the viral vaccine, TLR agonist and anti-CD40 antibody can be formulated together or separately with a pharmaceutically acceptable carrier for administration and prevention or treatment of a viral infection.

While the instant composition and methods find application in the prevention and treatment of viral infections of mammals, in particular humans, the invention should be construed to include administration to a variety of animals, including, but not limited to, cats, dogs, horses, cows, cattle, sheep, goats, birds such as chickens, ducks, geese, and fish.

An effective amount, as used in the context of the instant invention, is an amount which produces a detectable primary or memory CTL response, IFN-gamma production, or neutralizing antibody response to a viral vaccine thereby generating protective immunity against the viral pathogen. As such, an effective amount of the instant composition prevents the signs or symptoms of a viral infection, or diminishes viral pathogenesis so that viral infection is treated. Responses to administration can be measured by analysis of subject's vital signs or monitoring viral load, IFN-gamma production, CTL responses or neutralizing antibody responses according to established methods.

A composition of the present invention can be formulated according to known methods to prepare a pharmaceutically useful composition, whereby the active agents are combined in admixture with a pharmaceutically acceptable carrier. Suitable carriers and their formulation are described, for example, in Remington: The Science and Practice of Pharmacy, Alfonso R. Gennaro, editor, 20th ed. Lippincott Williams & Wilkins: Philadelphia, Pa., 2000.

Administration of a composition disclosed herein can be carried out by any suitable means, including parenteral injection (such as intraperitoneal, subcutaneous, or intramuscular injection), orally, or by topical application (typically carried in a pharmaceutical formulation) to an airway surface. Topical application to an airway surface can be carried out by intranasal administration (e.g., by use of dropper, swab, or inhaler which deposits a pharmaceutical formulation intranasally). As viral vaccines administered through a natural route of infection often induce local immunity, topical application to an airway surface offers certain advantages. In this regard, topical administration can be achieved by inhalation, such as by creating respirable particles of a pharmaceutical formulation (including both solid particles and liquid particles) containing the composition as an aerosol suspension, and then causing the subject to inhale the respirable particles. Methods and apparatus for administering respirable particles of pharmaceutical formulations are well-known, and any conventional technique can be employed. Oral administration can be in the form of an ingestable liquid or solid formulation.

Moreover, administration of each agent of the instant composition can be via the same or different route. For example, in the case of an inactivated influenza viral vaccine, both TLR agonist and anti-CD40 antibody can be injected by the same intradermal route, whereas in nasal administration of an attenuated influenza viral vaccine, the TLR agonist can be administered nasally and the anti-CD40 antibody can be administered intravenously or intradermally, as the site of action is believed to be the lymph nodes.

Administration can be given in a single dose schedule, or a multiple dose schedule in which a primary course of treatment can be with 1-10 separate doses, followed by other doses given at subsequent time intervals required to maintain and or reinforce the response, for example, at 1-4 months for a second dose, and if needed, a subsequent dose(s) after several months.

The exact dosage for administration can be determined by the skilled practitioner, in light of factors related to the subject that requires prevention or treatment. Dosage and administration are adjusted to provide sufficient levels of the composition or to maintain the desired effect of preventing or reducing viral signs or symptoms, or reducing severity of the viral infection. Factors which may be taken into account include the severity of the disease state, general health of the subject, age, weight, and gender of the subject, diet, time and frequency of administration, drug combination(s), reaction sensitivities, and tolerance/response to therapy.

When employing an anti-CD40 antibody in conjunction with a TLR agonist, it is contemplated that the dose of anti-CD40 can be reduced 10-fold or more over conventional doses given the efficacy of the TLR agonist for inducing an immune response.

The invention is described in greater detail by the following non-limiting examples.

### EXAMPLE 1

### Primary CTL Response

To establish the use TLR agonists and anti-CD40 antibody for facilitating maximal expansion of CD8+ T cells, C57BL/6 mice were immunized with 5 mg ovalbumin, ±50 μg anti-CD40 monoclonal antibody (FGK45.5; Rolink, et al. (1996) Immunity 5(4):319-30), ±10 mg/kg of a TLR7 agonist. Six days after injection, spleen cells were isolated and stained with anti-CD8-PE and for ovalbumin peptide (Ser-Ile-Ile-Asn-Phe-Glu-Lys-Leu tetramer; SEQ ID NO:11). Data from these experiments indicated that anti-CD40 antibody and TLR agonist increase tetramer-positive CTL by approximately 40 fold over background. Thus, CD40 and TLR agonists are essential for maximal expansion of CD8+ T cells to soluble peptide. Similarly, co-administration of M2₉₁₋₉₉ peptide of gammaherpesvirus-68 with CpG (TLR9 agonist) or anti-CD40 monoclonal antibody provided a 4-8 fold increase in CD8 CTL response (as determined by the number of cells synthesizing IFN-gamma) in mice as compared to injection with M2₉₁₋₉₉ peptide alone. Significantly, co-administration of CpG and anti-CD40 with M2₉₁₋₉₉ peptide provided a >100-fold synergistic increase in the generation of herpesvirus-68-specific IFN-gamma producing cells. It is contemplated that the primary mechanism of this enhancement is based upon the positive effects of TLR and anti-CD40 antibody stimulation on dendritic cell maturation. Coordinate with dendritic cell maturation, there are substantial increases in co-stimulatory molecule display and regulation of other processes of the class I and II MHC antigen processing pathways that combine to increase the effectiveness of the dendritic cell as the most important antigen-presenting cell for stimulating primary T cell responses.

Dose-response analysis of CTL response to MVA versus WR vaccinia virus was conducted. CTL lytic activity was determined using in vitro chromium release assays using effector cells taken directly from the immunized mice or subjected to an additional 6-day in vitro re-stimulation culture. It was found that in a secondary anti-vaccinia virus CTL response, MVA had a reduced immunogenicity compared to WR vaccinia virus (Table 4).

TABLE 4

| | % Specific Lysis | | |
|---|---|---|---|
| | 5 × 10⁶ pfu | 1 × 10⁶ pfu | 5 × 10⁵ pfu |
| Treatment | dose | dose | dose |
| WR (E:T = 4:1) | 100 | 107 | 99 |
| WR (E:T = 0.8:1) | 55 | 87 | 58 |
| MVA (E:T = 4:1) | 78 | 77 | 51 |
| MVA (E:T = 0.8:1) | 31 | 33 | 14 |

E:T, effector to target cell ratio.

In an analysis of primary anti-vaccinia virus CTL response to WR vaccinia virus or MVA, MVA also demonstrates reduced immunogenicity for the day 7 acute response. Accordingly, to determine whether immunogenicity of MVA could be enhanced, TLR agonist and anti-CD40 antibody were administered as a single injection at the time of vaccination with MVA to emulate a one-time immunization. MVA was co-administered with anti-CD40 monoclonal antibody and CpG DNA (i.e., TLR agonist). IFN-gamma ELISPOT analysis of spleen cells (TABLE 5) indicated that a single in vivo treatment with TLR9 agonist CpG DNA and anti-CD40 monoclonal antibody increased IFN-gamma producing recall response after infection with MVA to approximately >85% of that for WR vaccinia virus.

TABLE 5

| | # Spots per 5 × 10⁵ | | |
|---|---|---|---|
| | Spleen Cells | | |
| | 5 × 10⁵ pfu | 5 × 10⁴ pfu | 5 × 10³ |
| Treatment | dose | dose | pfu dose |
| WR vaccinia virus | 255 ± 65 | 167 ± 16 | 151 ± 8 |
| MVA | 103 ± 18 | 93 ± 44 | 61 ± 24 |
| MVA + CpG DNA/anti-CD40 | 234 ± 17 | 148 ± 38 | 139 ± 1 |

Moreover, a variety of TLR agonists including peptidoglycan (PGN; TLR2 agonist) unmethylated CpG DNA (1826; TLR9 agonist), and polyinosinic-polycytidylic acid (pIC; TLR3 agonist) in combination with anti-CD40 monoclonal antibody were found to augment MVA (1×10⁶ pfu) immunogenicity for a primary anti-vaccinia virus CTL response (Table 6). In particular, anti-CD40 monoclonal antibody and TLR9 agonist CpG (100 μg), caused an approximate 4-5 fold increase in the lytic activity of mice immunized with a dose of MVA at which it was significantly less immunogenic than WR vaccinia virus.

TABLE 6

| | % Specific Lysis | | |
|---|---|---|---|
| Treatment | E:T = 150:1 | E:T = 30:1 | E:T = 60:1 |
| MVA only | 3.1 | 2.1 | 0.225 |
| MVA + PGN + anti-CD40 | 7.4 | 5.0 | 1.7 |
| MVA + CpG + anti-CD40 | 11.7 | 10.1 | 1.07 |
| MVA + pIC + anti-CD40 | 5.8 | 2.6 | 0.7 |

Likewise, TLR agonists including lipopolysaccharides (LPS; TLR4 agonist) and pIC (TLR3 agonist) in combination with anti-CD40 monoclonal antibody were found to augment the immunogenicity of a 3×10⁶ pfu dose of MVA for a primary anti-vaccinia virus CTL response (Table 7).

TABLE 7

| | % Specific Lysis | | |
|---|---|---|---|
| Treatment | E:T = 150:1 | E:T = 30:1 | E:T = 60:1 |
| MVA only | 17 | 6 | 1 |
| MVA + LPS + anti-CD40 | 28 | 15 | 4 |
| MVA + pIC + anti-CD40 | 35 | 20 | 5 |

### EXAMPLE 2

### Memory T Cell Response

To demonstrate a memory T cell response, mice were infected with 2×10⁶ infectious units of MVA virus, and either 50 μg anti-CD40 monoclonal antibody alone or in combination with 100 μg of pIC (TLR3 agonist) or CpG DNA (TLR9 agonist). After 9.5 weeks, a memory T cell response, as determined by IFN-gamma ELISPOT analysis of spleen cells, was detected (Table 8).

TABLE 8

| Treatment | # Spots per 1 × 10⁵ Spleen Cells |
|---|---|
| MVA only | 15 |
| MVA + anti-CD40 Ab | 26 ± 4 |
| MVA + pIC + anti-CD40 Ab | 56 ± 8 |
| MVA + CpG + anti-CD40 Ab | 63 ± 5 |

Memory CTL production by spleen cells from mice infected with MVA for 7.5 weeks was also augmented by toll-like receptor agonists (Table 9). Mice receiving 2×10⁶ infectious units of MVA virus, and 50 μg anti-CD40 monoclonal antibody alone, or in combination with either 100 μg of PIC (TLR3 agonist) or CpG DNA (TLR9 agonist), or both PIC and CpG exhibited memory CTL production at levels equal to or slightly greater than mice infected in parallel with WR vaccinia virus.

TABLE 9

| | % Specific Lysis | |
|---|---|---|
| | | 6 Day in vitro |
| | No in vitro | Stimulation with |
| Treatment | Stimulation | WR Vaccinia Virus |
| MVA only | 2 | 24 |
| MVA + anti-CD40 | 0 | 32 |
| MVA + pIC + anti-CD40 | 5 | 49 |
| MVA + CpG + anti-CD40 | 0 | 68 |
| MVA + pIC + CpG + anti-CD40 | 1 | 52 |
| WR Vaccinia Virus | 14 | 57 |

To demonstrate that a Toll-like receptor agonist alone could elicit memory CD8⁺ T cell production of IFN-gamma, mice were concomitantly administered MVA and Imiquimod (a TLR 7 agonist). For this analysis, mice received 2×10⁶ infectious units of MVA virus, and either 50 μg anti-CD40 monoclonal antibody or 100 μg of Imiquimod. After 9.5 weeks, the mice were sacrificed, and standard intracellular cytokine staining techniques were employed, with spleen cells analyzed on a FACS CALIBUR flow cytometer. As demonstrated by the results provided in Table 10, a Toll-like receptor agonist was sufficient to induce memory CD8⁺ T cell production of IFN-gamma comparable to mice infected in parallel with WR vaccinia virus.

TABLE 10

| | % Total CD8⁺ Cells Expressing |
|---|---|
| Treatment | IFN-gamma |
| MVA only | 6.79 |
| MVA + anti-CD40 | 7.275 |
| MVA + Imiquimod | 15.694 |
| WR Vaccinia Virus | 21.51 |

### EXAMPLE 3

### Neutralizing Antibody Response

Neutralizing antibody responses were also analyzed. Mice were infected with MVA and administered a combination of TLR3 (pIC), TLR7 (Imiquimod), and/or TLR9 (CpG) agonists, with or without anti-CD40 monoclonal antibody. Serum was isolated and standard plaque inhibition assays were performed. Briefly, WR vaccinia virus-infected 143B cell cultures were pretreated with preimmune or MVA/anti-CD40/TLR agonist immune sera from mice infected for 7 days or 7.5 weeks with MVA, plaques were enumerated, and the percent inhibition was calculated. The percent of plaque inhibition for control WR vaccinia virus immune sera was consistently ˜90%. As demonstrated by the results provided in Table 11, a neutralizing antibody response was elicited by Toll-like receptor agonists in the presence and absence of an anti-CD40 monoclonal antibody.

TABLE 11

% Inhibition

| | 7 Day | 7.5 Week |
|---|---|---|
| | Post- | Post- |
| Treatment | Infection | Infection |
| None | 0 | |
| anti-CD40 | 0 | 0 |
| Imiquimod + anti-CD40 | 0 | 0 |
| pIC + anti-CD40 | 0 | 0 |
| CpG + anti-CD40 | 14.6 | 6.5 |
| pIC + Imiquimod + anti-CD40 | 0 | 17 |
| Imiquimod + CpG + anti-CD40 | 21.8 | 22.6 |
| pIC + CpG + anti-CD40 | 2.7 | 0 |
| pIC + Imiquimod + CpG + anti-CD40 | 0 | 22.6 |
| Imiquimod | 13 | 22.6 |

What is claimed is:

1. A composition comprising a viral vaccine and at least one Toll-like receptor agonist.

2. The composition of claim 1, further comprising an anti-CD40 antibody.

3. The composition of claim 1, wherein the Toll-like receptor is an intracellular receptor.

4. The composition of claim 1, wherein said composition comprises at least two Toll-like receptor agonists.

5. A method for increasing the immunogenicity of a viral vaccine comprising administering a viral vaccine in combination with at least one Toll-like receptor agonist thereby increasing the immunogenicity of the viral vaccine.

6. The method of claim 4, further comprising administering an anti-CD40 antibody.

7. A method for preventing or treating a viral infection comprising administering an effective amount of a viral vaccine in combination with at least one Toll-like receptor agonist so that a viral infection is prevented or treated.

8. The method of claim 7, further comprising administering an anti-CD40 antibody.
